(19) United States
(12) Patent Application Publication
(10) Pub. No.: US 2008/0255472 A1
(43) Pub. Date: Oct. 16, 2008

(54) Ovulation-prediction devices with image processing system

(76) Inventor: Youti Kuo, Penfield, NY (US)

Correspondence Address: Youti Kuo, 88 FOXBOURNE ROAD, PENFIELD, NY, 14526, US

(21) Appl. No.: 11/786,903

(22) Filed: Apr. 13, 2007

(51) Int. Cl. A61B 10/00

(52) U.S. Cl. 600/551; 382/128

(57) ABSTRACT

An ovulation-prediction device with an image processing system containing a transmissive test platen for placing saliva samples and a miniature camera for capturing the image of saliva sample at dried state for analyzing the increase of salt content for predicting ovulation. An image processing method with an algorithm described for removing noise in a saliva sample for calculating the density of dark pixels which represents salt content in the sample. A Density Index is defined. Trend curve of Density Index vs. day is established based on the daily saliva analysis in a woman's menstrual cycle for predicting days from the ovulation. Also illustrated are applications of the image processing system in an electrical toothbrush, a dental massager and a portable compact device using a disposable tape cassette for testing saliva samples.

### BACKGROUND OF THE INVENTION

There is a growing need for a home diagnostic system for monitoring various personal physiological conditions especially for the prediction of ovulation for women. A reliable method of predicting ovulation can determine a woman's fertility period for pregnancy as well as for birth control. A convenient oral device such as an electrical toothbrush or a portal device having a built-in ovulation-prediction capability is desirable for checking a woman's fertility condition on a daily basis.

(1) Field of the Invention

The present invention relates to handheld devices having an image processing system for analyzing the increase of salt content in dried saliva samples for the prediction of ovulation day.

(2) Related Art

It is known that a woman's menstrual cycle, in general, lasts from 27 to 30 days, while menstruation lasts from 3 to 7 days in the cycle. In each cycle a woman can conceive only during about a three to six day window. As a woman's fertile period lasts about six days and ends on the day of ovulation a fertility test based upon detection of ovulation on the ovulation day is too late to be useful in determining the fertility time for planning. For advanced prediction a urine test on the concentration of luteinizing hormone (LH) can detect ovulation 1-2 days ahead of time but the test is not sufficient to detect the entire fertile period of three to six day. There are many methods for predicting a woman's ovulation. It is known in the art to measure a woman's body temperature which increases with estrogen's rise to detect fertile times. It has been demonstrated that shortly after menstruation begins the body temperature decreases until ovulation starts, and after that the temperature increases. During the menstruation period, the vaginal secretions also becomes increasingly viscous and to peak at the time of maximum fertility. These body temperature and viscosity measurements methods, however, are not reliable in determining fertile periods. Other ovulation prediction methods include a blood test and an urine test for detecting a surge on estrogen-related hormone. These tests can determine whether the woman is at ovulation instead of providing advanced signal of impending ovulation.

Saliva is a complex body fluid containing several different electrolytes including salts of sodium, potassium chloride and non-electrolyte components including several proteins, enzymes, and immunoglobulins. U.S. Pat. No. 4,770,186 by Regas et al. uses a sensor probe for measuring the electrical resistance of a saliva sample. Daily measurements are made beginning not more than five days following the beginning of menstruation. The onset of ovulation is determined as a function of a peak electrical resistance measurement following the onset of menstruation. A Stage A peak of salivary electrical resistance (SER) occurs approximately six days, plus or minus one day, prior to ovulation. After a sharp dip following the Stage A peak, Stage B peak occurs approximately 2 days before ovulation, plus or minus one day and it is a sign of imminent ovulation. Although the trend of changing electrical resistance of these electrolytes in saliva can be used to predict the impending ovulation, the appearance of multiple peaks prior to ovulation is too complicated to make a reliable judgement. Furthermore, the signal level of the electrical resistance is generally too weak to enable an accurate prediction.

Specifically, several patents in prior art describe various methods for collecting and diagnosing the contents of saliva for the prediction of ovulation. U.S. Pat. No. 3,968,011 by Manautou et al. shows the use of the optical density curves of saliva samples to indicate pregnancy. Such curves have a first peak and a smaller second peak in daily measurements; however, the second peak is eliminated when pregnancy occurs. In application, a paper test strip impregnated with a peroxidase and guaiac shows a color change when wet with saliva during the fertile period. The change is caused by the presence of peroxide in the saliva. The test strip is costly and may not be reused. U.S. Pat. No. 4,385,125 by Preti et al. monitors saliva for the concentration of certain long-chain alcohols, particularly dodecanol, for detecting ovulation. The dodecanol content of saliva remains at a relatively constant level throughout the menstrual cycle, but exhibits a single peak at the time of ovulation. Because the method requires the use of an incubated saliva sample, it is more suitable for laboratory tests than home use. Also the fact that the dodecanol level exhibits a single peak or spike precisely corresponding to ovulation does not enable prediction of a fertile period ahead of ovulation necessary for planning. U.S. Pat. No. 5,914,271 by Law et al. discloses that a saliva's calcium and magnesium concentration drops in the three to five day period immediately prior to ovulation. It provides methods of monitoring the calcium and magnesium concentration. All the methods include using a reagent composition such as calcium or magnesium sensitive dye or pigments which undergoes a visible change in the presence of a clinically significant threshold concentration of the ion. However, the use of reagent for a test stripe, ion-selective electrodes, or a handheld reflectometer for detecting different color shades is inconvenient for regular home testing. Other commercially available handheld devices predict ovulation based on a measured peak in electrical resistance corresponding to sodium and potassium electrolyte levels which are reflective of hormone changes that occur several days before ovulation. The measured data on the changes of electrolytes in saliva may be inconsistent since an oral sensor probe is placed on the tongue where the thickness of the saliva layer may vary. While there are disadvantages associated with all of the above methods, each method demonstrates the feasibility of using an optical sensor or a conductivity sensor for measuring signals derived from a saliva sample to predict a fertile period or ovulation.

For predicting ovulation, a commercially available OV-Watch functions by detecting changes in the chemical composition of a woman's perspiration during sleep. Its bio-sensor detects chloride-ion levels from user's skin. Changes in the concentration of chloride-ions are detectable due to the increase or decrease of certain reproductive hormones like estrogen. This OV-Watch is inconvenient as it requires an user to wear the watch nightly while sleeping for monitoring the signal of the chloride-ion level for the prediction of ovulation.

Another method for determining the ovulation is by visual examination of a woman's dried saliva. The method is based on observations of crystallized salt pattern in a dried saliva, which is referred as ferning pattern. The physical basis of ferning pattern is not well known. Some research results correlate the crystallization pattern with increases in the chloride content, changes in ionic strength and/or the content of sodium or potassium in the saliva. Research results mentioned in U.S. Pat. No. 4,815,835 by Corona indicates that saliva crystallization appears when the blood folliculin level has reached a certain height that coincides with the third or fourth day before ovulation. The crystallization pattern is visible under 100-fold magnification of a saliva sample on a slide. The crystallization lasts until 3 or 4 days after ovulation, when the presence of lutein inhibits the crystallization. At fertile times, microscopic viewing of a dried saliva reveals a structure of salt distribution pattern that starts to form chains. This method of examination of saliva offers a reliable way to determine fertility. U.S. Pat. No. 5,572,370 by Cho describes an apparatus for determining the fertile periods of women based on laboratory observations of crystallized saliva under high magnification. When a woman is most fertile, the saliva dries in fern-like patterns and during non-fertile periods the saliva pattern is random and generally appears as unconnected dots. When a combination of dots and fern-like patterns appear, it indicates that the woman is in a transitional period that a conception is possible but not highly likely. The patent states that laboratory tests have shown the fern-like structures appearing approximately three to four days prior to ovulation and ending two to three days after ovulation. However, the described method relies on the experience of visual observations and comparisons with standard patterns for determination of the fertile and non-fertile conditions of the woman being tested, therefore, it is subject to inaccuracies. Also described in U.S. Pat. No. 5,639,424 by Rausnitz is a portable fertility tester for viewing the ferning pattern of a dried saliva sample. The tester has a circular disc with transparent regions indexed to each of the days of the menstrual cycle for storing the saliva patterns for viewing. An ocular is provided with a magnifying lens for examining the appearance of a-woman's saliva sample placed on the tester. After drying, a fernlike pattern indicates the woman at a fertile time or a structureless dotted pattern that indicates non-fertile. The ovulation tester was approved by FDA in January of 2002 (The Associate Press news article on Jan. 19, 2002). The device, however, depends solely on qualitative viewing of multiple stored saliva samples for determining the fertility condition and no quantitative trend is established for more accurate prediction of ovulation.

Instead of qualitative visual observations of crystalline patterns, U.S. Pat. No. 6,159,159 by Canter et al. describes an approach of ovulation monitoring by quantitatively determining the degree of ferning on the basis of diffraction of light by a crystallized saliva sample. A laser light is directed onto a targeted location on a dried sample that reflects scattered light onto a two-dimensional photo diode array. The photo diode array inputs the light intensity profile to a microprocessor. The microprocessor has a programmed algorithm that calculates a local ferning index representing a characteristic structure in the diffraction pattern of the targeted location. By this means, a number of locations are selected for obtaining a summary ferning index that represents the degree of ferning of the whole saliva sample. The approach uses a threshold value for determining the fertility of the saliva sample. The accuracy of this method, however, depends on selected measurement locations, which may not represent the whole imaged area of a dried saliva sample.

For predicting ovulation by capturing image of dried saliva, U.S. Pat. No. 6,960,170 of Kuo describes an image processing system using a test channel in brushhead for collecting saliva sample and a miniature camera for capturing the image of the saliva at dried state for analyzing the crystalline patterns. The system uses an algorithm that calculates the characteristic line length of line segments of connected saliva dots and uses a ferning index number for prediction ovulation day. Such an algorithm requires a large memory for a microprocessor and the use of the test channel is too complicated for collecting saliva sample. However, since the increased ferning pattern is a result of surge of potassium chloride or salt contents near ovulation, a direct monitoring of the increase of dark pixels representing salt contents can simplify the algorithm for predicting the ovulation day. Also, instead of using a test channel, the imaging of a saliva sample can be simplified by placing the saliva sample directly on an exposed test platen or on a disposable test tape which does not require cleaning. It is therefore the objective of the present invention to describe simpler means for placing a saliva sample for capturing image for testing without using a test channel and a simpler algorithm for calculating the increase of salt contents, which requires less microprocessor memory for monitoring and predicting ovulation.

### SUMMARY OF INVENTION

An embodiment of an oral device for home use is configured as an ovulation-prediction electrical toothbrush which has a handle and a brush head. The handle contains a clear test platen, battery, motor, a rotatable driveshaft, microprocessor, display, timer and a miniature digital camera. A plurality of bristles which rotate or oscillate are attached to the top of the brush head. The optic transmissive test platen is for placing a saliva sample for drying and the camera is for capturing an image of dried saliva sample and transmits the image to the microprocessor. An algorithm in the microprocessor analyzes the saliva image and calculates pixel density of dark image of salt contents to translate into a density index value for establishing a trend curve for predicting ovulation. The timer is used for ensuring that sufficient time is allowed for drying the saliva sample before an image of the saliva sample is captured by the camera. The test platen is cleaned for repeated uses for placing new saliva samples. Another embodiment is a portal device using a disposable tape cassette for placing saliva samples on an optic transmissive test tape. The test tape is advanced for exposing new area for placing a new saliva sample for testing that does not require cleaning after each testing.

In operation, after a predetermined drying time period, the camera captures the image of the dried saliva sample. A density index is computed based on the density of dark pixels of saliva dots appearing in the dried saliva sample. The growth data of the density Index in the preceding days of interest are plotted as trend curves and displayed in the display unit. Based on the trend curves a prediction of impending ovulation is provided.

For a portable ovulation-prediction device the essential components include; a) a digital camera assembly for capturing image saliva sample; b) a timer; c) a display; d) an image processing algorithm software for computing the Density Index of an image pattern of dried saliva sample; e) a microprocessor having a control program for the operation of the device; f) a power source; g) a tape cassette for providing test tape for placing saliva samples.

### BRIEF DESCRIPTION OF THE DRAWINGS

FIG. 1 is a side cross-section view of an ovulation-prediction electrical toothbrush.

FIG. 2 is a schematic longitudinal A-A cross section illustration of the camera system of the ovulation-prediction electrical toothbrush of FIG. 1.

FIG. 3a is an illustration of a random distribution of saliva dots in a dried saliva in a infertile time.

FIG. 3b is an illustration of the presence of connected saliva dots as line segments in dried saliva near the time of ovulation.

FIG. 3c is an illustration of the ferning pattern of structured line segments of saliva dots in dried saliva at the time of ovulation.

FIG. 4a illustrates a matrix of pixels in an image area of a dried saliva sample in a infertile time.

FIG. 4b illustrates a matrix of pixels in an image area of a dried saliva sample near the time of ovulation.

FIG. 4c illustrates a matrix of pixels in an image area of a dried saliva sample at the time of ovulation.

FIG. 5a illustrates a matrix of pixels of FIG. 4a with noise pixels being converted to white pixels.

FIG. 5b illustrates a matrix of pixels of FIG. 4b with noise pixels being converted to white pixels.

FIG. 5c illustrates a matrix of pixels of FIG. 4c with noise pixels being converted to white pixels.

FIG. 6 is a display of trend curve of dark pixel density near the time of ovulation.

FIG. 7 is a display of a trend curve of the Density Index near the time of ovulation.

FIG. 8 is a flow chart of image processing for calculating saliva Density Index and for the prediction of ovulation day.

FIG. 9 is a side cross-section view of a ovulation-prediction electrical gum massager.

FIG. 10a is an ovulation-prediction device with tape cassette detached from the imaging station housing.

FIG. 10b is a front view of the imaging station housing of the ovulation-prediction device of FIG. 10a.

FIG. 10c is a front view of the tape cassette of the ovulation-prediction device of FIG. 10a.

FIG. 11a is the ovulation-prediction device of FIG. 10a with tape cassette attached to the imaging station housing.

FIG. 11b is a cross-section view of the ovulation-prediction device of FIG. 11a.

FIG. 11c is a cross-section view of the ovulation-prediction device of FIG. 11a with the lid at closed position.

### DETAILED DESCRIPTION OF THE INVENTION

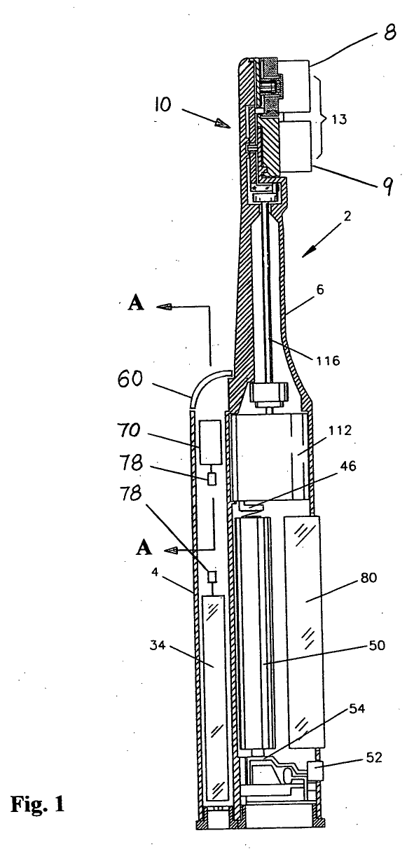

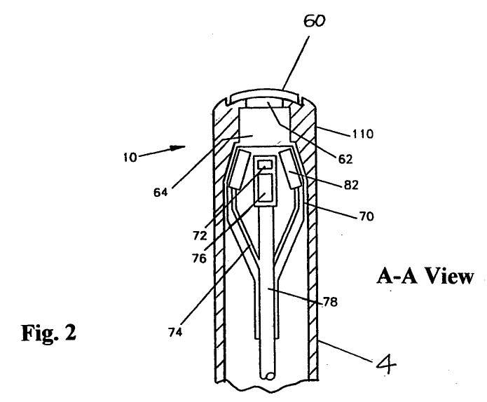

FIG. 1 shows an ovulation-prediction electrical toothbrush 2 having a handle 4 and a brushhead 10 connected by neck 6. Replaceable bristle unit 13 having rotary bristle element 8 and stationary bristle element 9 is detachably mounted on brushhead 10. Motor 112, batteries 50, microprocessor 34 and display 80 are positioned in handle 4. Leaf spring contact 54 is situated at the end of battery 50 and switch 52 extends through an opening in the base of the handle. Batteries 50 are connected to motor 112 by contact 46. Drive shaft 116, having a central longitudinal axis with first end and second end, is positioned in neck 6. The replaceable bristle unit 13 is engaged with an oscillation linkage with a straight lever contained in brushhead 10. Referring to FIG. 1 and FIG. 2, handle 4 in FIG. 1 has an external surface for supporting an optic transmissive test platen 60 and cavity 64 underneath for housing camera assembly 70 and microprocessor 34. A saliva sample can be placed directly on top of transmissive platen 60 for drying and for image capture by the camera assembly. Also, the transmissive test platen 60 may be of ellipsoid shaped dome for satisfying optimized imaging requirements for the imaging chip of camera assembly 70 and illumination sources 82, which are positioned in the proximity of the focal point of the shape of the test platen. Test platen 60 may be repeatedly cleaned for testing new saliva samples.

Further shown in FIG. 2, preferably the camera assembly 70 has a charge coupled device (CCD) 76 having a two dimensional photosensor array, an optic assembly 72 for focusing on image area 62 on optically transmissive platen 60 and an illumination assembly 74 providing targeted illumination to the image area 62. Camera assembly 70 is positioned such that it has the desired field of view and is focused on the image area 62 of the platen 60 for transmitting signals representative of the image received from the image area to display system 80 by cable 78. In addition, The illumination assembly 74 includes a light guide and a light source, both located entirely inside the camera assembly 70. The light guide 82 has a terminal end aiming at the image area 62 of the test platen 60 and a source end in communication with the light source. Power for the light source is communicated into the camera assembly by the cable 78. The digital camera 70 is adapted to capture the image area 62 upon an activation signal. The activation signal is provided by the microprocessor 34 which has a programmed timing depending on the drying status set by an user or by a default value. The binary image data output from the image sensing CCD 76 is provided to the microprocessor 34, which includes a data acquisition component. The microprocessor generates pixel data representing the coordinates of the image pixels. The camera 70 is miniaturized for fitting into the handle. A CCD having diameter less than 5 mm is commercially available. Optionally, the camera assembly may utilize a CMOS photo diode array in place of the CCD type of photosensor array. A CMOS imaging sensor with sensing area 4.55 mm×2.97 mm and pixel size of 6.0 micron square is commercially available.

The microprocessor has a random access memory (RAM) unit and a programmable read only memory (PROM) unit. The RAM unit contains programming related to the operation of the electrical components and the PROM contains algorithm software for sensor signal calibration and image processing. The information stored in RAM unit is read through an I/O. Moreover, at the same time it triggers a timer (not shown) inside handle 4 in communication with microprocessor 34. Following a predetermined time period at the plateau state of dried saliva sample, the control program of the microprocessor activates the camera assembly to capture the image of the dried saliva.

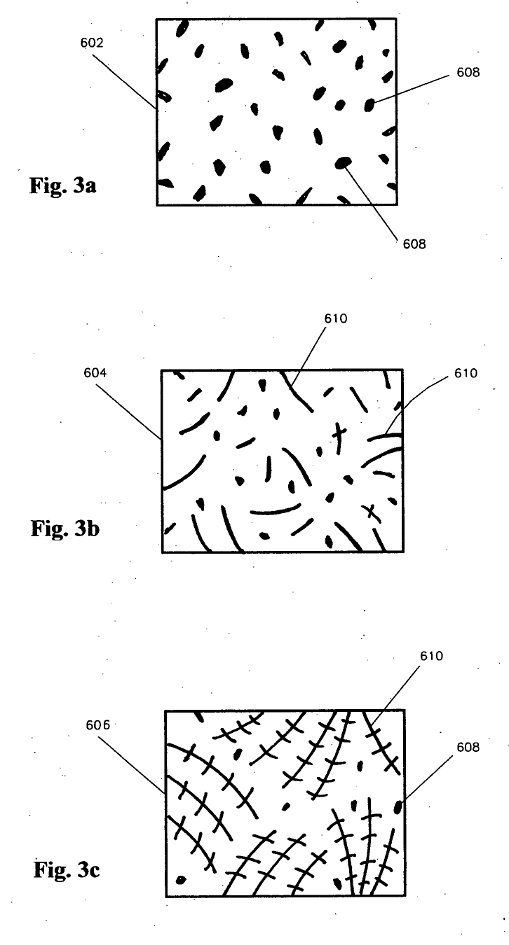

FIGS. 3a, 3b, 3c show typical images 602, 604, 606 of a woman's dried saliva in infertile and fertile periods of a menstrual cycle. Physically the structured features as illustrated are related to the connectivity of saliva dots in the crystallization process. FIG. 3a illustrates a random distribution of unconnected saliva dots 608 during an infertile time when long-chain alcohol is at a constant level. FIG. 3b illustrates a partial crystalline pattern 604 with the presence of a significant degree of connected saliva dots or line segment 610 indicating the transition period. The increase of long-chain alcohol, particularly dodecanol near the time of ovulation results in the linking of the saliva dots that appear in line segments. The connectivity and the length of saliva dots increase to the maximum at the time of ovulation as shown in a nearly full crystalline pattern 606 in FIG. 3c. In the present invention a single parameter, the density of dark pixels, is directly measured for evaluating the surge of potassium chloride or salts contents associated with the degree of crystallization or structured line pattern which is observed near the day of ovulation. The change of density of dark pixels during a menstrual cycle can be illustrated by a trend curve. The peak of the trend curve predicts the timing of ovulation.

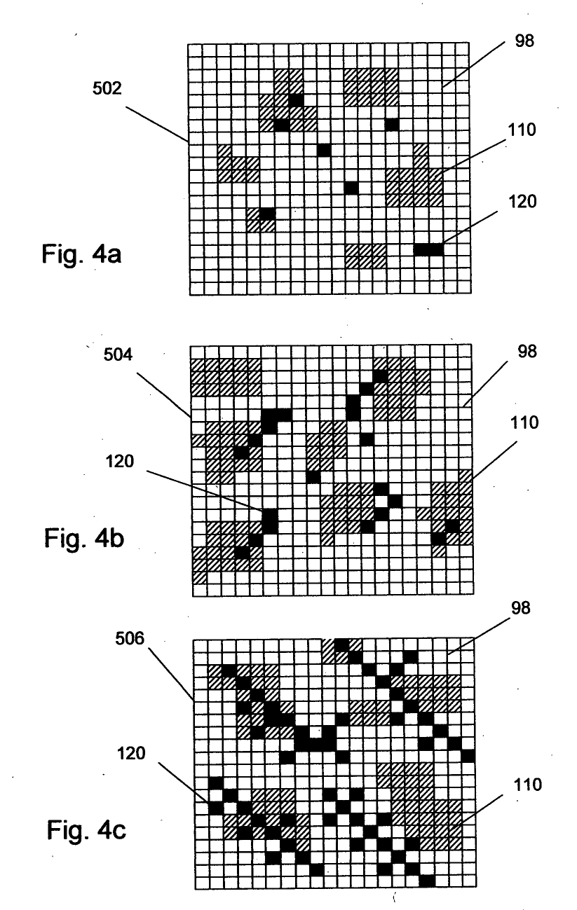

A method of automated image processing of digitizing a digital image for the evaluation of dark pixel density includes the following steps: (1) storing the test image pattern of the dried saliva sample, (2) determining gray levels of dark pixels, background and noise pixels, (3) converting the background and noise pixels to white pixels, and (4) calculating the density of dark pixels as well as plotting trend curves of the Density Index. FIG. 4a represents a matrix 502 of image pixels taken from a dried saliva sample at an infertile time. FIG. 4b represents a matrix 504 of image pixels taken from a dried saliva sample at a transition time. And FIG. 4c represents a matrix 506 of image pixels taken from a dried saliva sample at the time of ovulation.

As shown in FIGS. 4a, 4b and 4c, the digitized image of targeted image area 62 captured by camera assembly 70 (shown in FIG. 2) contains a number of pixels with each pixel having a three-dimensional information space. An image pixel has a two-dimensional array representing X and Y coordinates and the third dimension indicating the gray level of the pixel. The gray level of each pixel is represented by three ranges of optical density which are out of 256 gray-scale values between black and white. The first range of optical density, D_{B}, includes the optical density of the background of transmissive platen 60, which is presented in light pixels 98. The second range of optical density, D_{N}, includes the optical densities of noise, such as air bubbles and contaminants, represented by hatched pixels 110, present in the saliva sample. The third range of optical density, D_{D}, includes the optical density of the saliva dots image 608, which is presented in dark pixels 120 so distinctively different from the noise and the background material of the transmissive platen. Dark pixels 120 reflecting the imaged saliva dots have a greater gray-scale value. The noise pixels 110 of air bubbles and “contaminants” are light pixels of lesser gray-scale values. And the pixels 98 of the transmissive platen are of least gray-level value. An optical pattern recognition program for digitizing a digital image detects these ranges of the optical density. By comparing the gray values of all the pixels, the optical pattern recognition program can identify the optical density range at D_{D} as dark pixels and assign “ones” as “black” and assign the optical density ranges at D_{B} and D_{N} as “zeros” or “white” in a binary system.

In practice, the gray level D_{B} of the transmissive test platen is obtained from an image of the test platen without having a saliva sample on top. The gray level of D_{N} is obtained from an image of a saliva sample taken at infertile time, in which only few dark pixels of salt dots are present. By excluding the peak optical density of the dark pixels from the saliva sample of the infertile time, the gray level range D_{N} of the noise and “contaminants” can be determined. Consequently, by converting the pixels of optical density levels of D_{B} and D_{N} into white pixels, only the dark and white pixels present in the converted image of a saliva sample.

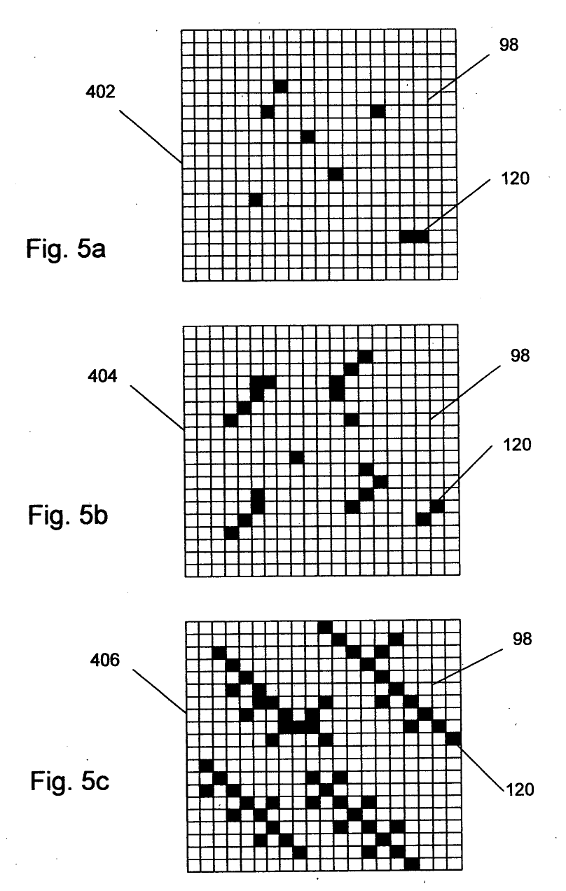

By following the above steps of filtering the background and noise pixels from an image of saliva sample, the dark pixels 120 of salt contents or dots in pixel matrix 502, 504 and 506 of FIGS. 4a, 4b and 4c remain as shown in pixel matrix 402, 404 and 406 in FIGS. 5a, 5b and 5c respectively, with background pixels and noise pixels being converted to white pixels. After filtering out the noise and the background, image of dried saliva samples can be used for calculating the density of dark pixels. The density of dark pixels is the ratio of the number of dark pixels to the number of total pixels in the image area as shown in the following first formula:

Density of Dark Pixels=(Number of dark pixels in the image area)/(Number of all pixels in the image area)

Where the number of all pixels in the image area is the sums of the number of dark pixels and the number of white pixels.

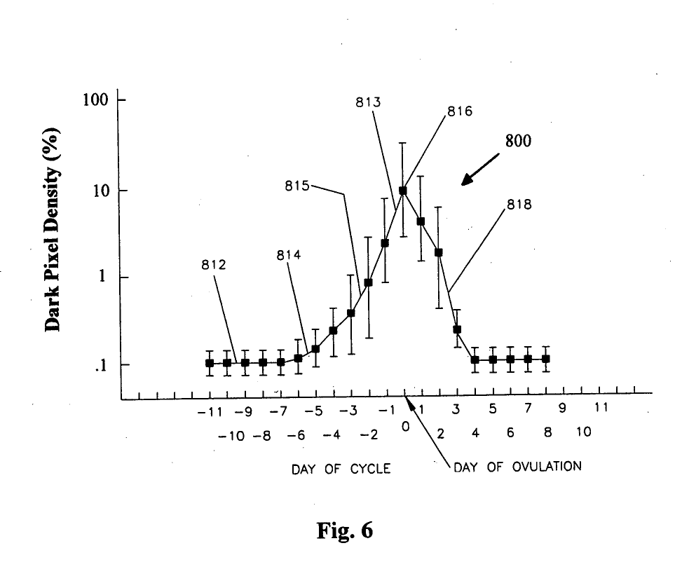

FIG. 6 is a plot of trend curve 800 of density of dark pixel vs day near the time of ovulation. The initial low level at flat portion 812 of the density of dark pixel curve indicates the random distribution of saliva dots 602. The initial increase portion as denoted 814 indicates the starting of crystallization which is about six or five days prior to ovulation. The growth of the crystalline pattern in the next few days reflects the increase of salt dots connected into line segments 610 as shown in dark pixels in FIG. 3b. At this transition stage as indicated in rapid growth portion 815, ovulation is expected to happen in two to one day. Eventually the salt content reaches a peak portion denoted by 816, on the day of ovulation. At that time a very structured crystalline pattern appears as shown in FIG. 3c. Thereafter, the salt content and the number of dark pixels starts to decrease as indicated by decline portion 818 of the trend curve, which drops to the same initial low level as indicated in 812 as in the infertile days.

For convenience, with a reference density a Density Index is defined as a percentage number for indicating the degree of reaching the maximum salt content. In the present invention, the Density Index is defined by the following second formula:

Density Index=(Current Density of Dark Pixel)/(Peak Density of Dark Pixel)

Where the Peak Density is an empirical value used as a reference density derived from the mean value of peak density data of a population of women. It may be optionally determined by a user's own peak density values occurred in preceding months. The peak of the Density Index curve which is near the value of “1” indicates the day of ovulation. However, for predicting the day of ovulation, the rate of increase of the Density Index curve indicates the impending ovulation in advance.

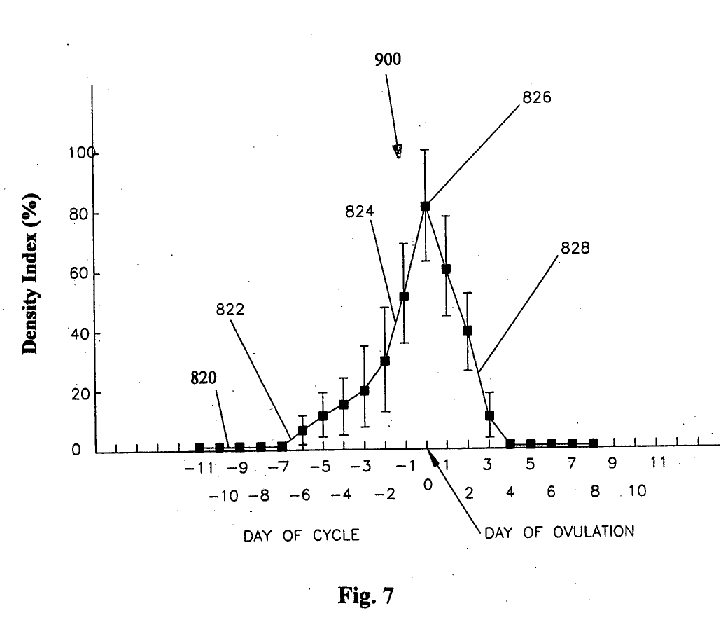

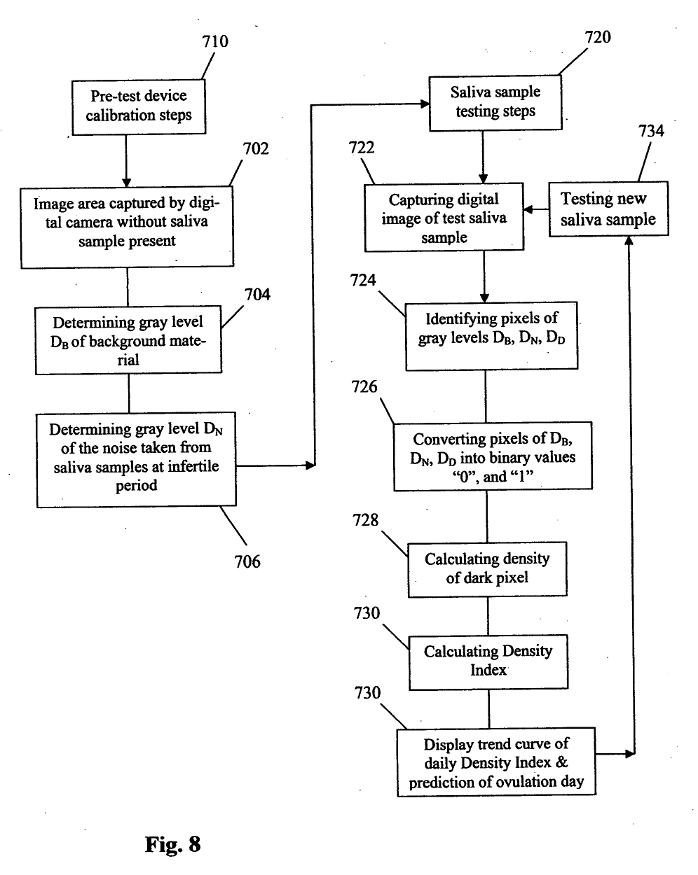

FIG. 7 shows a trend curve 900 of the Density Index near the time of ovulation. The trend curve portion 820 near zero on infertile days corresponds to the density of dark pixel 812 shown in FIG. 6. Similarly, rise portion 822, growth portion 824, peak portion 826 and decline portion 828 of trend curve 820 are corresponding to their counterparts, 814, 815, 816, and 818 respectively in trend curve 800 of the density of dark pixel as shown in FIG. 6. As a summary, referring to FIG. 8, the process of calculating saliva Density Index and the prediction of ovulation includes pre-test device calibration steps 710 and saliva sample testing steps 720. The calibration steps 710 comprise; (1) step 702 capturing an image of test platen without saliva sample present, (2) step 704 determining the background gray level D_{B} of test platen, (3) step 706 determining gray level D_{N} of the noise of an image of a saliva sample taken at infertile period. The saliva sample testing steps 720 comprise; (4) step 722 capturing a digital image of a saliva sample, (5) step 724 identifying pixels of three gray levels D_{B}, D_{N} and D_{D}, (5) step 726 converting pixels of gray levels D_{B} and D_{N} into white pixels of binary value “0” and pixels of gray level D_{D} into dark pixels of binary value “1”, (6) step 728 calculating density of dark pixels with binary value “1”, (7) step 730 calculating Density Index, (8) step 732 displaying the trend curve of daily Density Index and prediction of ovulation day, and (9) step 734 testing new saliva sample on succeeding day.

After a period of drying time for ensuring the drying of a saliva sample to be tested, image of the dried saliva sample is captured by the digital camera situated inside the handle. Stored image data of saliva dots are distinguished between light and dark pixels. Based on the binary information of saliva dots the computations of Density Index of the imaged area of dried saliva sample are performed. Repeated daily imaging of saliva samples and computations of Density Index enable plotting of a trend curve for display and providing prediction of impending ovulation. After each saliva testing for ovulation, the test platen is cleaned for next placement of new saliva sample for testing.

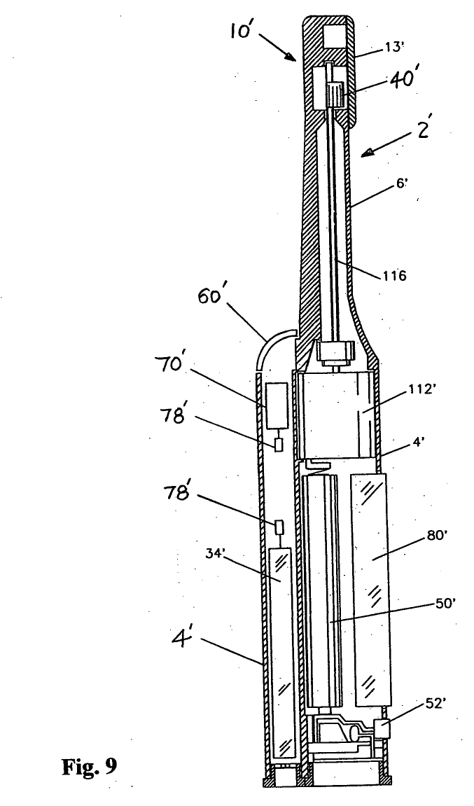

Instead of in the handle of a toothbrush, the test platen and the imaging system for saliva ovulation monitoring can be built in any device which is convenient for daily use. FIG. 9 shows a side cross-section view of a ovulation-prediction electrical oral device having a gum massager. The ovulation-prediction oral device 2′ having handle 4″ and a gum massager head 10′ connected by neck 6′. Motor 112′ and batteries 50′ are positioned within handle 4′. Switch 52′ extends through an opening in the base of the handle for activating the operation of the oral device. Drive shaft 116′, having a central longitudinal axis with first end engaged with the motor and second end mounted with biased wheel 40′ for imparting the vibration motion when the motor is turn on. Massager head 10′ has a dimple rubber layer 13′ for contacting with gums for vibration stimulation and camera assembly 70′ for capturing image of a dried saliva through optically transmissive platen 60′ in the handle. Microprocessor 34′ which is in communication with camera assembly 70′ through cable 78′ is for the control of the electrical components and the calculations of the Density Index. In addition, display 80′ is for the display of trend curve of Density Index as described previously.

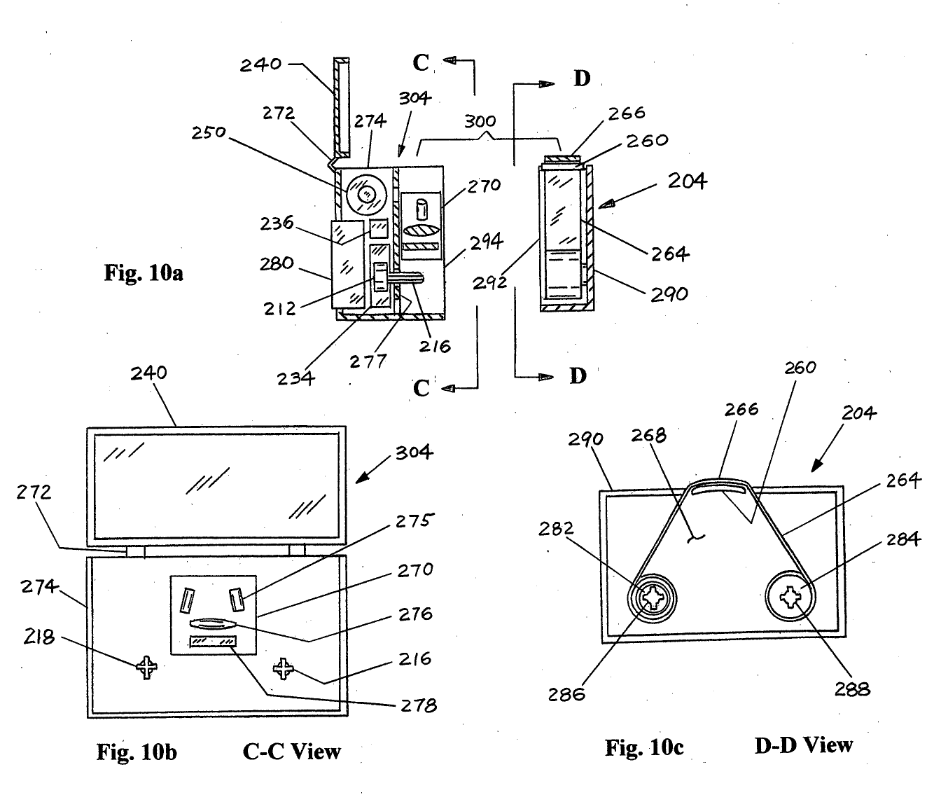

The proceeding ovulation-prediction devices as described are to be used normally at home where water is readily available for cleaning of the test platen after testing of saliva samples for repeated uses. Optionally, an ovulation-prediction system of the present invention may be included in a portable device using a tape cassette for advancing a tape segment as test platen. The tape cassette is disposable, therefore, it does not require cleaning after testing of a saliva sample. A portable ovulation-prediction device of the present invention includes an imaging station for imaging and analysis of saliva samples, and a detachable tape cassette having a platen and a test tape for placing saliva samples for testing. The platen and test tape are optic transmissive. FIG. 10a shows an ovulation-prediction portable device 300 having an imaging station 304 and a detachable tape cassette 204 which contains an optic transmissive platen 260 and an optic transmissive test tape 264. The imaging station housing 304 comprises a camera assembly 270, display 280, microprocessor 234, timer 235, battery 250, motor 212, shafts 216 and 218 as well as lid 240 which is attached to the housing 274 by hinges 272. FIG. 10b shows a front C-C view of image station 304 housing with lid 240 in open position and FIG. 10c shows a front D-D view of the tape cassette 204.

In FIG. 10c, tape cassette 204 of the ovulation-prediction device 300 comprises a transmissive platen 260 and transmissive test tape 264, which is wound on a pair of reels 282, 284 having drive slots 286, 288. The rotatable reels 282, 284 and the platen 260 are supported by cassette shell 290. The tape 264 is spanned over the platen 260 with its head end fastened to the head reel 284 and its tail fastened to the tail reel 282. The platen 260 is of partial convex surface that enables close contact between the tape test area 266 and the platen surface when the tape is tightened by a drive mechanism. The space 268 beneath the test platen 260 and between the two reels 282, 284 is an exposed recess area accessible from the interface side 292 of the tape cassette 204 for accommodating the camera assembly 270 when the tape cassette 204 is mounted on the cassette holder of imaging station housing 274. Referring to FIG. 10b, the camera assembly 270 and drive shafts 216, 218 are on the interface side 294 of the imaging station 304, exposed for the engagement with the tape cassette 204.

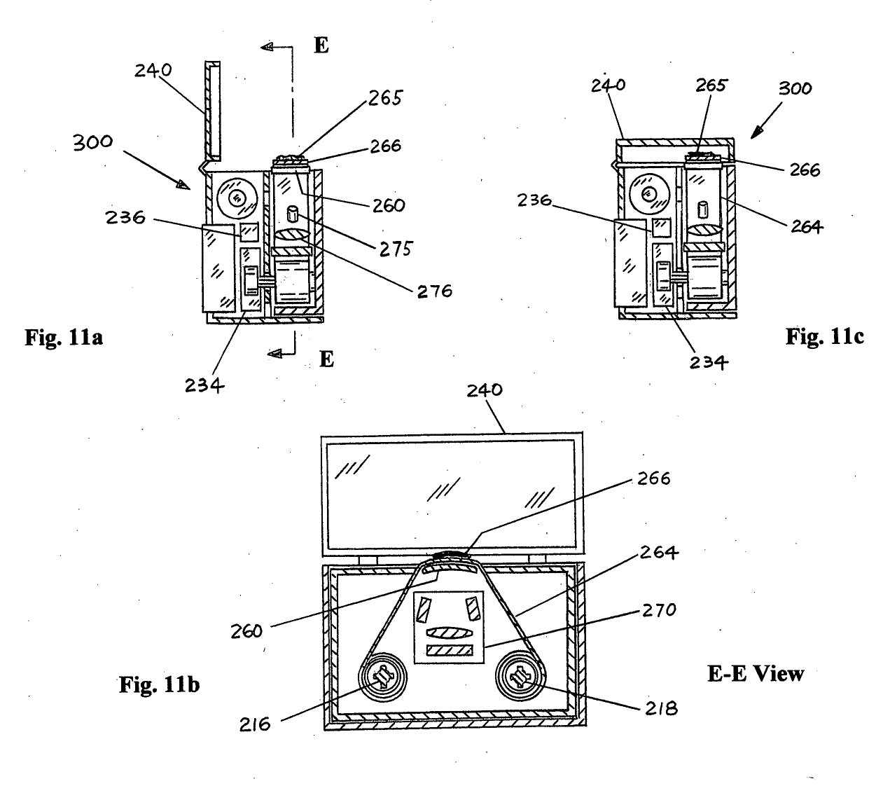

When the tape cassette 204 being mounted as shown in FIG. 11a, drive slots 286, 288 of tape reels 282, 284 are engaged with drive shafts 216, 218 in the housing 274. Test tape 266 supported by platen 260 is positioned at a distance equal to the focus distance above the lens 276 for capturing image of saliva sample 265 placed on the test tape 266 by the camera 278. The drive shafts 216, 218 extend from the partition wall 277 and they are in communication with motor 212. The camera assembly 270 and the motor are in communication with the microprocessor for operation control. A control circuit for controlling a tape transport mechanism detects the amount of the tape remaining in the tail reel.

FIG. 11a shows the lid 240 in open position and a saliva sample 265 being placed on the top of the test tape 266. A cross-section view E-E of FIG. 11a is shown in FIG. 11b. For capturing an image of the saliva sample 265, the lid 240 is closed on top of the test tape 266 as shown in FIG. 11c. The lid is designed to form gaps and vent grooves (not shown) at the closed position for air circulation for facilitating drying of the saliva sample. For ensuring the drying of a saliva sample prior to capturing an image of the saliva by the camera, a drying time with the lid closed on top of the clear tape is preset for the timer 236, which is in communication with the microprocessor 234. This venting function is required for users who want to place a saliva sample and then immediately close the lid for viewing the test result at later time. The preset drying time for the timer is determined at the lid closed condition. After capturing the image of the saliva sample, the test tape is automatically advanced or indexed for a segment to have a new tape surface for placing next saliva sample for testing. This automatic tape indexing mechanism is included in the control circuit in the microprocessor. The image processing steps of this portable device is the same as that described in the preceding sections for the ovulation-prediction toothbrush as shown in FIG. 8.

In a preferred embodiment of an imaging system of a portal device of FIGS. 10a 10b, 10c, the light source comprises a solid state light emitting diode (LED) 275 and the imaging camera 278 is of a chip-type CMOS (complementary metal oxide semiconductor). The optical system includes lens 276 for focusing light on to the CMOS imaging camera.

The present invention has been described in detail with reference to preferred embodiments thereof. However, variations and modifications can be implemented within the spirit and scope of this invention. Instead of processing the image data by the internal microprocessor, the digital image signal input from the digital camera can be transmitted by wireless signal transmitting circuit to a computer which is loaded with an imaging processing software for analysis. Furthermore, the image of a dried saliva sample may be optionally transmitted by optical fibers to a digital camera positioned external to the handle of a device as described by the present invention. The use of optical fibers for transmitting optical image is well known in the art.

I claim:

1. An image processing system for predicting ovulation of a female comprising: a. means for providing a digital image of a dried saliva sample obtained from the female; b. image processing means for calculating pixel density of saliva dots in an image area of the digital image of a dried saliva sample; and c. means for predicting ovulation based on an increase of said pixel density of saliva dots.

2. An image processing system for predicting ovulation of a female of claim 1, wherein said image processing means comprises: a means for digitizing the digital image of the dried saliva sample by identifying saliva dots with dark pixels, background and noise with light pixels; and b. means for excluding light pixels from calculating pixel density of saliva dots.

3. An image processing system for predicting ovulation of a female of claim 2, wherein said image processing means includes steps of; a) identifying the gray level D_{D} of saliva dots and converting the pixels of saliva dots into dark pixels, b) identifying the gray level D_{B} of the transmissive test platen from an image of the test platen without having a saliva sample, c) identifying the gray level D_{N} of noise from an image of a saliva sample taken at infertile time, d) converting the pixels of optical density levels of D_{B} and D_{N} into light pixels.

4. An image processing system for predicting ovulation of a female of claim 2, wherein said image processing means comprises means for calculating a Density Index, wherein said Density Index is based on a percentage of pixel density of saliva dots to a reference density.

5. An image processing system for predicting ovulation of a female of claim 4, wherein the increase of said Density Index is displayed as a trend curve.

6. An image processing system for predicting ovulation of a female of claim 1, wherein said means for providing a digital image of a saliva sample comprises: a. test platen for placing said saliva sample, b. a digital camera for capturing a digital image, c. a microprocessor in communication with the digital camera, d. a display for displaying outputs of said image processing means, e. a power source.

7. An image processing system for predicting ovulation of a female of claim 6, wherein the test platen is optic transmissive.

8. An image processing system for predicting ovulation of a female of claim 7, wherein the test platen is placed on the handle of an electrical toothbrush.

9. An image processing system for predicting ovulation of a female of claim 6, wherein the test platen is placed on the handle of an electrical massager.

10. A device for predicting ovulation of a female comprising:
a. a digital camera assembly comprising an image sensor, a lens and a light source,
b. a display
c. a microprocessor in communication with said digital camera assembly and display,
d. a power source,
e. a tape cassette comprising a tape spanned over a test platen,
f. a device housing for supporting said camera assembly, display, microprocessor and power source and being adaptive for the attachment of said tape cassette.

11. A device for predicting ovulation of a female of claim 10, wherein said tape and test platen of said tape cassette are optic transmissive.

12. A device for predicting ovulation of a female of claim 10 including an image processing system for predicting ovulation of a female comprising; a. means for providing a digital image of a dried saliva sample obtained from the female; b. image processing means for calculating pixel density of saliva dots in an image area of the digital image of a dried saliva sample; and c. means for predicting ovulation based on an increase of said pixel density of saliva dots.

13. A device for predicting ovulation of a female of claim 12, wherein said image processing means comprises: a. means for digitizing the digital image of the dried saliva sample by identifying saliva dots with dark pixels, background and noise with light pixels; and b. means for excluding light pixels from calculating pixel density of saliva dots.

14. A device for predicting ovulation of a female of claim 13, wherein said image processing means comprises means for calculating a Density Index, wherein said Density Index is based on a percentage of pixel density of saliva dots to a reference density.

15. A device for predicting ovulation of a female of claim 14, wherein the increase of said Density Index is displayed as a trend curve.
